(19) United States
(12) Patent Application Publication
(10) Pub. No.: US 2004/0242506 A1
(43) Pub. Date: Dec. 2, 2004

(54) Paroxetine glycyrrhizinate

(76) Inventors: Nathalie Claude Marianne Barges Causeret, Mayenne (FR); Nicola Lisa Anna Marzolini, Harlow (GB); Padma Meneaud, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/486,468

(22) PCT Filed: Aug. 9, 2002
(86) PCT No.: PCT/EP02/08926

(30) Foreign Application Priority Data
Aug. 9, 2001 (GB) 0119467.9

(51) Int. Cl. A61K 31/704; A61K 31/205

(52) U.S. Cl. 514/33; 514/554; 536/18.1

(57) ABSTRACT

A salt formed from paroxetine hydrochloride and ammonium glycyrrhyzinate masks the bitter taste of paroxetine and has a distinctive liquorice flavour.

The present invention relates to a novel compound, to processes for preparing it and to its use in treating medical disorders.

Pharmaceutical products with antidepressant and anti-Parkinson properties are described In U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylenedioxy-phenoxymethyl)-piperidine. This compound is used in therapy as the hydrochloride salt for the treatment and prophylaxis of inter alia depression, obsessive compulsive disorder (OCD) and panic.

We have now surprisingly discovered a novel salt of paroxetine with glycyrrhyzinic acid which may be used as an alternative to the currently marketed hydrochloride.

According to the present invention there is provided paroxetine glycyrrhyzinate as a novel compound.

A great advantage of the glycyrrhyzinate salt in oral formulations is its intense flavour of sweet liquorice which provides a taste-masking effect to hide the bitterness of paroxetine.

In fact, because of the intensity of the liquorice flavour, further flavourings may be desirable to modify the liquorice taste of the formulation.

In one aspect the novel salt of this invention is provided in non-crystalline form, which may a solid or an oil. The oil is preferably absorbed on a solid carrier, especially a carrier that is usable as a component of a pharmaceutical composition.

In another aspect the novel salt of this invention is provided in crystalline form. When the crystalline form exists as more than one polymorph, each polymorph forms another aspect of this invention.

Paroxetine glycyrrhyzinate may be prepared by contacting stoichiometric amounts of the acid and paroxetine free base. Preferably the base is in solution, more preferably both are in solution.

Most commonly used solvents are suitable for mobilising paroxetine free base, for example toluene, alcohols such as methanol, ethanol, propan-2-ol, esters such as ethyl acetate, ketones such as acetone and butanone, halogenated hydrocarbons such as dichloromethane, and ethers such as tetrahydrofuran and diethyl ether. The glycyrrhyzinic acid is preferably added as an aqueous or etahnolic solution. The glycyrrhyzinic acid may also be added in the form of a soluble salt, for example ammonium glycyrrhyzinate, or the glycyrrhyzinic acid salt of an amine, for example ethylamine or diethylamine.

The concentration of paroxetine base is preferably in the range 5 to 50% weight/volume, more preferably in the range 10 to 30%. The concentration of glycyrrhyzinic acid is suitably in the same range. Elevated temperatures may be used to increase solubility.

The salt may be isolated in solid form by conventional means from a solution thereof obtained as above. For example, a noncrystalline salt may be prepared by precipitation from solution, spray drying and freeze drying of solutions, evaporating a solution to a glass, or vacuum drying of oils, or solidification of melts obtained from reaction of the free base and the acid.

A crystalline salt may be prepared by directly crystallising from a solvent in which the product has limited solubility, or by triturating or otherwise crystallising a non-crystalline salt. An improved yield of the salt is obtained by evaporation of some or all of the solvent or by crystallisation at elevated temperature followed by controlled cooling, preferably in stages. Careful control of precipitation temperature and seeding may be used to improve the reproducibity of the production process and the particle size distribution and form of the product. Individual polymorphs are preferably crystallized directly from a solution of the salt, although recrystallizing a solution of one polymorph using seeds of another polymorph may also be carried out.

An alternative method of preparing paroxetine glycyrrhyzinate is to start with a salt of paroxetine with an organic acid, such as acetic acid or maleic acid, rather than using paroxetine free base. Use of another salt of paroxetine as a starting material is suitable for preparation of the crystalline salt or, if a volatile acid such as acetic acid is used, non-crystalline salts by methods that involve evaporation (such as freeze-drying and spray-drying).

We ahv found it particularly efecive to combine paroxetrine hydrochloride with ammonium glycyrrhyzinate.

The salt may obtained as a solvate, when during isolation from solution it becomes associated with the solvent in which it is dissolved. Any such solvate forms a further aspect of this invention. Solvates may be returned to the unsolvated salt by heating, for example by oven-drying, or by treatment with a displacement solvent which does not form a solvate.

Prior to the isolation of the paroxetine glycyrrhyzinate, water may be removed from the solution containing the salt by azeotropic distillation to avoid the formation of hydrates or to obtain the product in anhydrous form. In that case, suitable solvents for the solution of the salt are those which form an azeotrope with water such as toluene and propan-2-ol. It should also be appreciated that mixtures of solvents can also be used to aid the azeotropic removal of water.

Paroxetine free base may be prepared according to the procedures generally outlined in U.S. Pat. No. 4,007,196 and EP-B-0223403. Glycyrrhyzinic acid is commercially available as the mono-ammonium, disodium and dipotassium salts.

The compounds of this invention may be used to treat and prevent the following disorders:

| | |
|---|---|
| Alcoholism | Anxiety |
| Depression | Obsessive Compulsive Disorder |
| Panic Disorder | Chronic Pain |
| Obesity | Senile Dementia |
| Migraine | Bulimia |
| Anorexia | Social Phobia |
| Pre-Menstrual Syndrome (PMS) | Adolescent Depression |
| Trichotillomania | Dysthymia |
| Substance Abuse | |

These disorders are herein after referred to as “the Disorders”.

The present invention further provides a method for treating and/or preventing any one or more of the Disorders by administering an effective and/or prophylactic amount of a salt of the invention to a sufferer in need thereof.

The present invention further provides a pharmaceutical composition for use in the treatment and/or prevention of the Disorders which comprises an admixture of a salt of the invention with a pharmaceutically acceptable carrier.

The present invention also provides the use of a salt of the invention for treating and/or preventing the Disorders.

The present invention also provides the use of a salt of the invention in the manufacture of a medicament for treating and/or preventing the Disorders.

Most suitably the present invention is applied to the treatment of depression, OCD and panic.

Compositions containing the salt of this invention may be formulated for administration by any route, and examples are oral, sub-lingual, rectal, topical, parenteral, intravenous or intramuscular administration; Preparations may, if desired, be designed to give slow release of the paroxetine salt.

The medicaments may, for example, be in the form of tablets, capsules, sachets, vials, powders, granules, lozenges, reconstitutable powders, or liquid preparations, for example solutions or suspensions, or suppositories.

The composition is usually presented as a unit dose composition containing from 1 to 200 mg of paroxetine calculated from the amount of salt on a free base basis, more usually from 5 to 100 mg, for example 10 to 50 mg such as 10, 12.5, 15, 20, 25, 30 or 40 mg by a human patient. Most preferably unit doses contain 20 mg of paroxetine calculated on a free base basis. Such a composition is normally taken from 1 to 6 times daily, for example 2, 3 or 4 times daily so that the total amount of active agent administered is within the range 5 to 400 mg of paroxetine calculated on a free base basis. Most preferably the unit dose is taken once a day.

The compositions of the invention are usually adapted for oral administration; preferred unit dosage forms include tablets or capsules.

The compositions of this invention maybe formulated by conventional methods of admixture such as blending, filling and compressing.

Suitable carriers for use in this invention include a diluent, a binder, a disintegrant, a colouring agent, a flavouring agent and/or preservative. These agents may be utilized in conventional manner, for example in a manner similar to that already used for marketed anti-depressant agents.

Specific examples of pharmaceutical compositions include those described EP-B-0223403 and U.S. Pat. No. 4,007,196, in which the products of the present invention maybe used as the active ingredients.

The following Examples illustrate the present invention:

### EXAMPLE 1

### Preparation of Tablets

| INGREDIENTS | 20 mg Tablet | 30 mg Tablet |
|---|---|---|
| Paroxetine Glycyrrhyzinate | 20.00 mg | 30.0 mg |
| | (calc. as free base) | (calc. as free base) |
| Dicalcium Phosphate (DCP) | 83.34 mg | 125.0 mg |
| Microcrystalline Cellulose | 50.67 mg | 76.0 mg |
| Sodium Starch Glycollate | 8.34 mg | 12.5 mg |
| Magnesium Stearate | 1.67 mg | 2.5 mg |

Commercial source of the ingredients

| | |
|---|---|
| Dicalcium Phosphate Dihydrate | Emcompress or Ditab* |
| Microcrystalline Cellulose | Avicel PH 102* |
| Sodium Starch Glycollate | Explotab.* |

*Trade names

Method

1. Pass DCP through a screen and weigh it into a Planetary mixer.

2. Add 30 mesh Paroxetine Glycyrrhyzinate to the bowl.

3. Add 20 mesh Avicel and Explotab and mix all the powders for 10 minutes.

4. Add magnesium stearate and mix for 5 minutes.

Tablet into Pentagonal Tablets Using the Following Punches:

| | | |
|---|---|---|
| 30 mg Tablet | 9.5 mm | Circumcircle |
| 20 mg Tablet | 8.25 mm | Circumcircle |

The tablets are made satisfactorily on a single punch or a Rotary press.

### EXAMPLE 2

### Preparation of Tablets

| INGREDIENTS | 10 mg Tablet | 20 mg Tablet | 30 mg Tablet |
|---|---|---|---|
| Paroxetine | 10 mg | 20 mg | 30 mg |
| Glycyrrhyzinate | (calc. as free base) | (calc. as | (calc. as |
| | | free base) | free base) |
| Sodium Starch | 2.98 mg | 5.95 mg | 8.93 mg |
| Glycollate | | | |
| Granular Dicalcium | | | |
| Phosphate | 158.88 mg | 317.75 mg | 476.63 mg |
| (DITAB) or Dicafos | | | |
| Magnesium Stearate | 1.75 mg | 3.50 mg | 5.25 mg |

Method

1. Paroxetine Glycyrrhyzinate, Sodium Starch Glycollate and Dicalcium Phosphate Dihydrate are screened and mixed together in a suitable mixer. (Planetary, Cuble or High Energy Shear mixer.)

2. Add Magnesium Stearate and compress it into a tablet using a single punch or Rotary Tablet machine.

What is claimed is:

1. A paroxetine glycyrrhyzinate salt.

2. A compound according to claim 1 in non-crystalline form.

3. A compound according to claim 1 in crystalline form.

4. A process for the preparation of a compound as claimed in claim 1 by precipitation from a solution of a paroxetine glycyrrhyzinate, spray drying or freeze drying a solution of a paroxetine glycyrrhyzinate, evaporating a solution of a paroxetine glycyrrhyzinate to a glass, or by vacuum drying of oils of a paroxetine glycyrrhyzinate, or solidification of melts of a paroxetine glycyrrhyzinate.

5. A process for the preparation of a compound as claimed in claim 1 by crystallization or re-crystallization from a solution of a paroxetine glycyrrhyzinate.

6. A process according to claim 4 or in which the solution, oil or melt of a paroxetine glycyrrhyzinate is prepared by treating paroxetine free base or an organic acid salt thereof with glycyrrhyzinic acid or an ammonium or amine salt thereof.

7. A method for treating and/or preventing any one or more of the Disorders by administering an effective and/or prophylactic amount of a paroxetine glycyrrhyzinate to a sufferer in need thereof.
